(19) United States
(12) Patent Application Publication
(10) Pub. No.: US 2023/0200878 A1
(43) Pub. Date: Jun. 29, 2023

(54) SYSTEMS AND METHODS FOR USING A MULTI-PROBE INTEGRATED ELECTROTHERMAL MODULES (ETMS) DEVICE FOR TUMOR ABLATION

(71) Applicant: Bard Peripheral Vascular, Inc., M/C 110 IP Ops, NJ (US)

(72) Inventors: Aseem Singh, Tempe, AZ (US); Oladipo Peter Akerele-Ale, Phoenix, AZ (US); Ruben Ramirez, Mesa, AZ (US); Eric Moll, Gilbert, AZ (US); Danielle Eldred, Tempe, AZ (US)

(73) Assignee: Bard Peripheral Vascular, Inc., M/C 110 IP Ops, NJ (US)

(21) Appl. No.: 17/995,336

(22) PCT Filed: Apr. 9, 2020
(86) PCT No.: PCT/US2020/027369

§ 371 Date: Oct. 3, 2022

(51) Int. Cl. A61B 18/08

(52) U.S. Cl. CPC A61B 18/082; A61B 2018/00577

(57) ABSTRACT

Systems and methods for tumor ablation with controlled precision of a temperature profile utilizing a tumor ablation probe device may include disposing a distal end of the tumor ablation probe device in a tissue, the distal end including a plurality of electrothermal modules (ETMs) on probe arm(s), each ETM including a first surface component electrically connected to a second surface component; supplying a first voltage of a first polarity or a second voltage of a second polarity to at least one ETM, and repeatedly alternating between the first polarity and the second polarity based on a time sequence cycle. When the first polarity is supplied, the ETM heats the first surface component and cools the second surface component, and when the second polarity is supplied, the ETM cools the first surface component and heats the second surface component. Each ETM and/or probe arm is configured for independent control.

### TECHNICAL FIELD

The present specification generally relates to medical devices for treating cancer, and more particularly to medical devices configured and operable for tumor ablation in a treatment area within a patient's body to achieve tumor necrosis.

### BACKGROUND

Common treatments for addressing tumor growth include invasive surgical resection techniques and minimally invasive ablation techniques. Radiofrequency (RF) treatment is an accepted ablation techniques. In general, RF treatment may face difficulties while attempting complete tumor destruction. Further, RF treatment may result in imprecisely defined tumor margins that affect areas of tissue beyond a site targeted for ablation, such as heat transfer to surrounding healthy areas of tissue, along with other potential complications with respect to probe setup within a disease region. Similar complications may occur for other treatment methods that cause direct (thermal) or indirect thermal (microwave) ablation.

Accordingly, a need exists for tumor ablation systems and methods with precise control over a temperature profile at an affected site to enhance tumor necrosis while maintaining excellent tumor margins to ablate unhealthy tissue with respect to the affected site avoiding external areas.

### SUMMARY

In one embodiment, a system for tumor ablation with controlled precision of a temperature profile utilizing a tumor ablation probe device may include the tumor ablation probe device, and a circuit controller communicatively coupled to the tumor ablation probe device and a non-transitory computer storage medium. The distal end may include a plurality of electrothermal modules (ETMs) proximally disposed on a device surface. Each ETM may include a first surface component and a second surface component opposite and electrically connected to the first surface component. The non-transitory computer storage medium stores instructions that, when executed by the circuit controller, cause the system to supply, via the circuit controller, one of a first voltage of a first polarity and a second voltage of a second polarity opposite the first polarity to at least one ETM of the plurality of ETMs, and repeatedly alternate, via the circuit controller, between the first polarity and the second polarity based on a time sequence cycle. When the first polarity is supplied, the at least one ETMs heats the first surface component and cools the second surface component. When the second polarity is supplied, the at least one ETM cools the first surface component and heats the second surface component. Each ETM may be configured for independent control by the circuit controller.

In one other embodiment, a method for tumor ablation with controlled precision of a temperature profile utilizing a tumor ablation probe device may include disposing a distal end of the tumor ablation probe device in a tissue, the distal end comprising a plurality of electrothermal modules (ETMs) proximally disposed on a device surface. Each ETM may include a first surface component and a second surface component opposite and electrically connected to the first surface component. The method may further include supplying, via a circuit controller communicatively coupled to the tumor ablation probe device, one of a first voltage of a first polarity and a second voltage of a second polarity opposite the first polarity to at least one ETM of the plurality of ETMs. When the first polarity is supplied, the at least one ETM heats the first surface component and cools the second surface component, and when the second polarity is supplied, the at least one ETM cools the first surface component and heats the second surface component. The method may further include repeatedly alternating, via the circuit controller, between the first polarity and the second polarity using on a time sequence cycle. Each ETM may be configured for independent control by the circuit controller.

In another embodiment, a method for tumor ablation with controlled precision of a temperature profile utilizing a tumor ablation probe device may include disposing a distal end of the tumor ablation probe device in a tissue, the distal end comprising at least one electrothermal module (ETM) on a first probe arm and at least one ETM on a second probe arm, and supplying, via a circuit controller communicatively coupled to the tumor ablation probe device, one of a first voltage of a first polarity and a second voltage of a second polarity opposite the first polarity to the at least one ETM on the first probe arm, the at least one ETM on the second probe arm, or both, as one or more voltage-supplied ETMs. Each ETM may include a first surface component and a second surface component opposite and electrically connected to the first surface component. When the first polarity is suppled, the one or more voltage-supplied ETMs respectively heats the first surface component and cools the second surface component. When the second polarity is supplied, the one or more voltage-supplied ETMs respectively cools the first surface component and heats the second surface component. The method may further include repeatedly alternating, via the circuit controller, between the first polarity and the second polarity using on a time sequence cycle. Each of the first probe arm and the second probe arm may be configured for independent control by the circuit controller.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a side perspective view of a tumor ablation probe device with multiple probes including integrated electrothermal modules (ETMs), according to one or more embodiments shown and described herein;

FIG. 2 is a side view of the tumor ablation probe device of FIG. 1 being used to treat a tumor, according to one or more embodiments shown and described herein;

FIG. 3 is a side elevation view of an embodiment of the tumor ablation probe device of FIG. 1, according to one or more embodiments shown and described herein;

FIG. 4 is a side view of another embodiment of a catheter of another tumor ablation probe device including balloon openings, according to one or more embodiments shown and described herein;

FIG. 5 is a side view of a needle with balloons including ETMs to use with the catheter of FIG. 4, according to one or more embodiments shown and described herein;

FIG. 6 is a side view of another tumor ablation probe device including a needle with integrated ETMs mounted thereon, according to one or more embodiments shown and described herein;

FIG. 7 is a side view of another tumor ablation probe device including a ribbed cone with integrated ETMs, according to one or more embodiments shown and described herein;

FIG. 8 is a side view of a catheter including an opening from which the ribbed cone of FIG. 7 is configure to flare out or retract into, according to one or more embodiments shown and described herein;

FIG. 9 is a perspective view of an integrated electrothermal module (ETM), according to one or more embodiments shown and described herein;

FIG. 10 is a flowchart of a process for using the tumor ablation probe device systems of FIGS. 1-9, according to one or more embodiments shown and described herein;

FIG. 11 is a flowchart of another process for using the tumor ablation probe device systems of FIGS. 1-9, according to one or more embodiments shown and described herein; and

FIG. 12 schematically illustrates a system for implementing computer and software based methods to utilize the tumor ablation probe device systems of FIGS. 1-9, according to one or more embodiments shown and described herein.

### DETAILED DESCRIPTION

Referring generally to the figures, embodiments of the present disclosure are directed to systems and methods for tumor ablation utilizing tumor ablation probe devices as described herein. Various embodiments of such systems and methods are described in detail herein.

Tumor ablation is a minimally invasive procedure that may be used to treat tumors such as of the liver, lung, kidney, and bone. During tumor ablation, thermal energy is used to heat or cool surrounding tissue to cytotoxic levels such as less than −40 degrees Celsius or greater than 60 degrees Celsius. Tumor ablation may use modalities such as radiofrequency (RF), thermal, microwave, chemical treatment, interventional radiology, laser, high-intensity focused ultrasound, localized radiation, cryoablation using extreme cold to destroy tissue (e.g., cause tissue necrosis) associated with a tumor, and irreversible electroporation. Such treatments may not achieve complete tumor destruction, particularly when tumor margins are not well defined. Furthermore, complications may occur from ablation of surrounding healthy tissue.

For the tumor ablation probe devices described herein, electrothermal modules (ETMs) are integrated at a distal end of a probe or catheter and, via thermal modulation (e.g., switching between heating/cooling by the same device), tumor necrosis is achieved within modulated tumor ablation margins. ETMs may be integrated on multiple probes housed within an overall needle/probe design. Such individual, individually movable, and individually controlled probes are advanced into the tumor and, when activated, invoke a heat and cryo cycling to cause tumor necrosis within precise tumor margins.

The ETMs may be thermoelectric generators, such as Seebeck generators. Seebeck generators convert temperature differences directly into electrical energy (e.g., through a Seebeck effect phenomenon in which a temperature differential between two electrically connected junctions produces an electromagnetic force between the junctions). Seebeck generators may operate in reverse such that applying a voltage to the device can cause it to act as a heater or cooler, depending on the magnitude and polarity of the voltage (e.g., though a Peltier effect phenomenon in which voltage applied across two electrically connected junctions produces a temperature differential between the junctions).

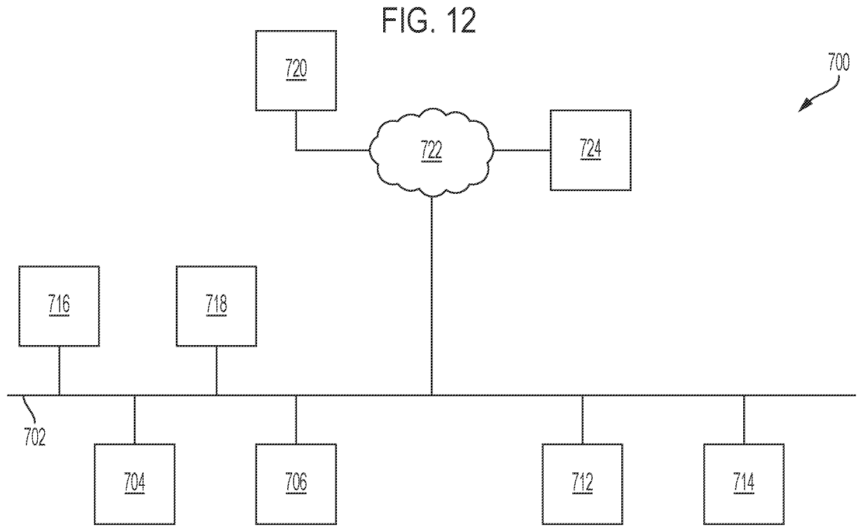

ETM integrated devices included in the devices, systems, and methods as described herein may precisely control an amount of heat generated and provide a rapid thermal/cooling cycling directly to a tumor to enhance tumor necrosis while maintaining tumor margins through precision temperature control on a focused area and while minimizing a risk to surrounding healthy tissue. The tumor ablation probe devices described herein may include a plurality of independently movable and controllable probe arms, each including one or more ETMs made of p-n couples and associated circuitry (e.g., through a processor 704 as described in greater detail below with respect to FIG. 12) configured to repeatedly alternate between hot and cold to cause tumor necrosis within precise, temperature controlled margins. For example, the ETMs may be placed around probe arms of a probe device, which probe arms may individually extend into a tumor region. In embodiments, the ETMS may be spaced about probe arms of the probe device, integrated onto probe arms of the probe device, or combinations thereof. A handle may track the depth to which a probe is inserted into the tumor.

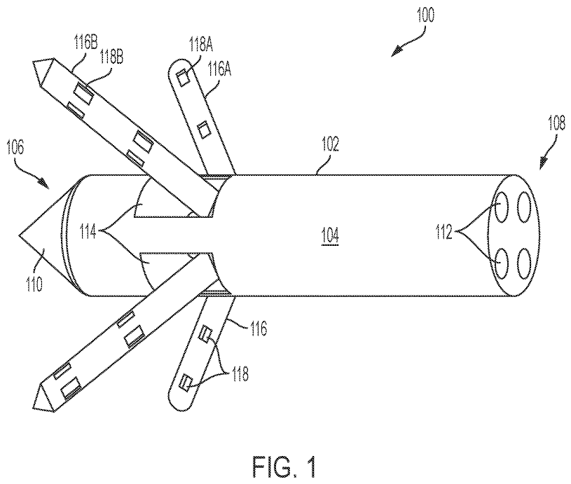

Referring to FIG. 1, a tumor ablation probe device 100 includes a probe 102 having an intermediate surface 104, a distal end 106, and a proximal end 108. The distal end 106 includes a tip 110 that is configured to pierce into a tumor. The intermediate surface 104 includes a plurality of openings 114 defined thereon. While the plurality of openings 114 are shown as concentrically and evenly spaced about the intermediate surface 104, the plurality of openings 114 may be disposed at uneven and/or staggered locations about the intermediate surface 104. Alternatively, the tumor ablation probe device 100 may include one opening 114. The proximal end 108 includes a plurality of apertures 112 configured to receive and house a respective plurality of probe arms 116. Each probe arm 116 is respectively configured to extend from or retract into each opening 114 of the intermediate surface 104 individually and/or independently.

Each probe arm 116 includes one or more integrated electrothermal modules (ETMs) 118. Each probe arm 116 may include a distal pierce portion configured to pierce a portion of a tumor. In embodiments, the tumor ablation probe device 100 may be semi-rigid, rigid, catheter based, or a similar type that is controlled via a handheld device including a controller circuit (e.g., through a processor 704 as described in greater detail below with respect to FIG. 12) configured to switch polarity of input power to provide to the integrated ETMs 118. Such a repeatedly altered switching of polarity on a predetermined time sequence cycle corresponds to and affects a pre-determined cycling of heating and cooling cycles of the ETMs 118 that are introduced into a tumor site in a minimally invasive manner to result in tissue necrosis. The tumor ablation probe device 100 may be battery powered and/or may utilize a voltage supplier to provide the input power.

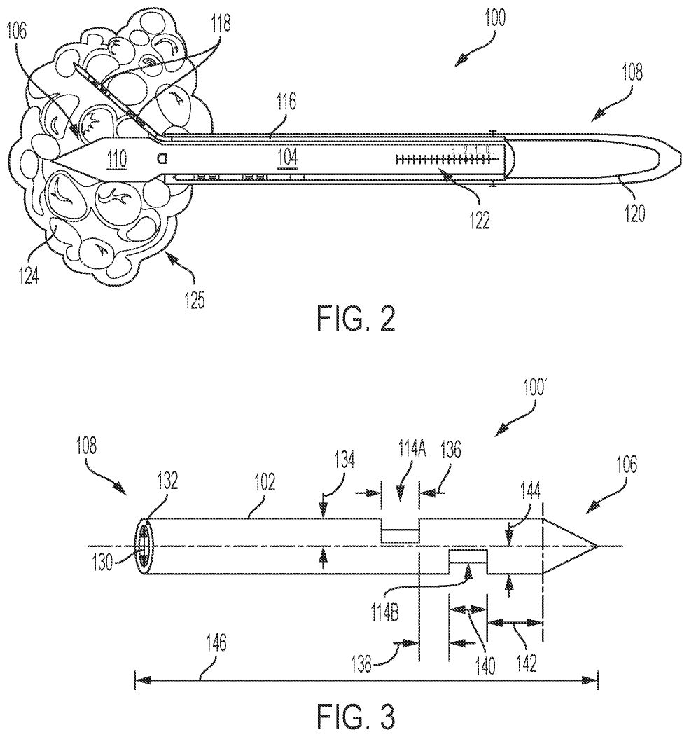

Referring to FIG. 2, the tumor ablation probe device 100 is shown as being advanced into a tumor 124 at a tumor site 125. The tumor ablation probe device 100 further includes a handle 120 including a metric display component 122 to track the advancement or retraction of a probe arm 116 including ETMs 118 into the tumor 124 when the tip 110 is disposed within the tumor 124. The metric display component 122 may measure such an advancement in centimeters, millimeters, inches, or other customary unit of length. Thus, the metric display component 122 provides a measurement system that may be associated with each individual probe arm 116 to determine a length the probe arm 116 has been extended or retracted. In embodiments, one or more metric display components 122 may be included on the tumor ablation probe device 100. By way of example, and not as a limitation, each probe arm 116 may include a respective metric display component 122.

Referring to FIG. 3, an embodiment of a tumor ablation probe device 100′, similar to the tumor ablation probe device 100 except for differences described herein, is shown. The tumor ablation probe device may include a plurality of openings 114 that include staggered openings 114A and 114B. In an embodiment, the tumor ablation probe device 100′ includes at the proximal end 108 an opening with a diameter 130 of 6.0 mm and a wall thickness 132 of 1.0 mm. The opening with the diameter 130 of the probe device 100′ extends into the probe device 100′ as an inner lumen, which also includes a diameter of 6.0 mm. The tumor ablation probe device 100′ further includes a first half portion 134 and a second half portion 144 of the intermediate surface 104 separated along a longitudinal axis. The staggered openings 114A and 114B include respectively opening lengths 136, 140 that may be of 4.5 mm each and staggered by a distance 138 of 2.0 mm between nearest ends. Respective probe arms 116 (FIG. 2) may be configured for extension through respective staggered openings 114A, 114B. The staggered openings 114A, 114B may be longitudinally staggered, angularly staggered, axially staggered, radially staggered, or combinations thereof. The staggered openings 114A, 114B may be angularly offset from one another, such as at a 180 degree offset. The staggered openings 114A, 114B may be configured with a size that is less than half of the circumference of the probe device 100′. In an embodiment, the staggered opening 114B closest to the distal end 106 is spaced at a distance 142 of 10 mm from the beginning of the tip 110 of the distal end 106. The tumor ablation probe device 100′ may include a length 146 between the proximal end 108 and the tip 110 of the distal end 106 of 200 mm.

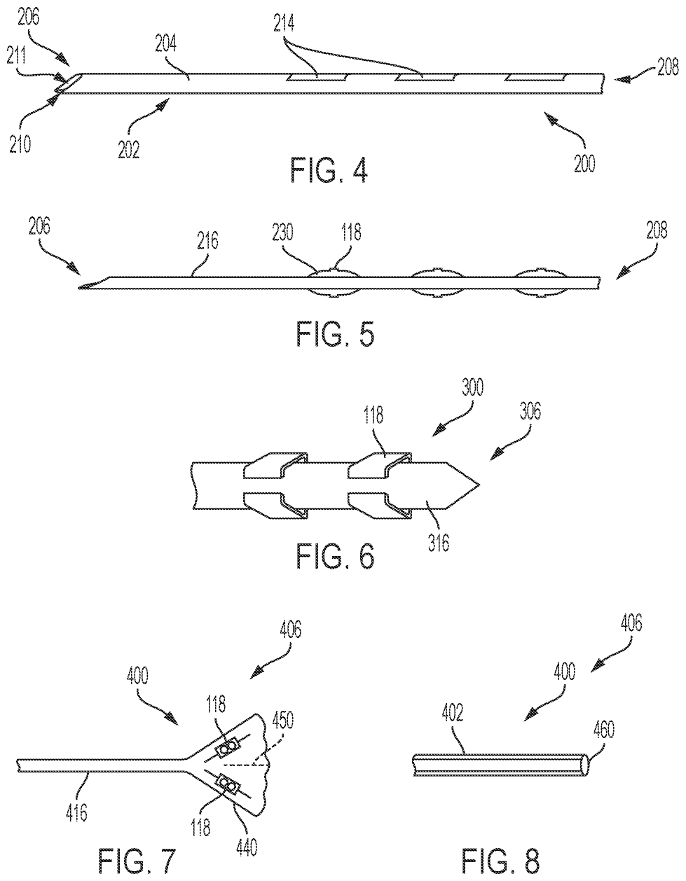

Referring to FIGS. 4 and 5, ETMs 118 are attached to balloons 230 that are configured to expand out through respective balloon openings 214 of a tumor ablation probe device 200. The tumor ablation probe device 200 illustrated in FIG. 4 includes a probe 202 to encase a needle 216 (e.g., as an embodiment of a probe arm) of FIG. 5. It is to be understood that reference to a needle within this disclosure is a reference to a probe arm as described herein, as the needle is an embodiment of the probe arm. The needle 216 includes a plurality of balloons 230 on which one or more ETMs 118 are disposed. While six balloons 230 are shown in the embodiment of FIG. 5 evenly disposed on opposite sides, it is understood within the scope of this disclosure that a different number of balloons and/or balloons having a different spacing may be utilized. In embodiments, approximately two to five ETMs 118 may be placed at various locations of each balloon 230. The needle 216 may be made of stainless steel. The probe 202 may be alternatively be a catheter. The probe 202 may be made of stainless steel.

The probe 202 includes an intermediate surface 204 disposed between a distal end 206 of the probe 202 and a proximal end 208 of the probe 202. The intermediate surface 204 includes a plurality of balloon openings 214 sized and shaped to permit advancement and/or retraction of each respective balloon 230 of the needle 216. The distal end 206 includes a tip 210 extending from the intermediate surface 204. A needle housing opening 211 is defined by an edge of the intermediate surface 204 and at least a portion of the tip 210 and extends as an inner lumen between the distal end 206 and the proximal end 208. The needle housing opening is configured to house the needle 216 in a position such that the plurality of balloons 230 are aligned with the respective plurality of balloon openings 214. In embodiments, a tip of the needle 216 may extend beyond an end of the tip 210, may be movable within the needle housing opening 211, may still within the needle housing opening 211, or combinations thereof.

During use, the tumor ablation probe device 200 is advanced into a tumor at a tumor site. When the balloons 230 are inflated at a pressure, the balloons 230 advance through respective balloon openings 214, and the ETMs 118 on each balloon 230 thus protrude along with the balloons 230 through the plurality of balloon openings 214 to sit flush with the tumor at the tumor site. Electrical energy is supplied to the ETMs 118 as described in greater detail below such that the ETMs 118 process through a heating/cooling cycle that is manually, automatically, or partially automatically controlled. Such localized heating/cooling causes necrosis of the tumor or tissue that the ETMs 118 directly contact in a precisely controlled manner within desired tumor margins. Thus, precise control over a temperature profile at an affected tumor site may be achieved through the tumor ablation probe devices described herein, such as the tumor ablation probe device 200. The tumor ablation probe devices described herein are suitable for minimally invasive use to further more effectively provide precisely controlled tumor necrosis. Upon release of pressure, the plurality of balloons 230 deflate to retract back into the plurality of balloon openings 214 along with the ETMs 118.

Referring to FIG. 6, in another embodiment, ETMs 118 are mounted to a needle 316 near and proximal to a distal end 306 of a tumor ablation probe device 300. When the tumor ablation probe device 300 is advanced into a tumor such that the ETMs sit flush with the tumor, energy supplied to the ETMs 118 provides a heating/cooling cycle as described further below to cause necrosis of the tumor.

Referring to FIGS. 7 and 8, ETMs 118 are mounted on a ribbed cone 440 that is configured to flare out from or retract into an opening 460 of a catheter 402 (e.g., as an embodiment of a probe) of a tumor ablation probe device 400 at a distal end 406. In embodiments, the ETMs 118 may be mounted on an external surface of the ribbed cone 440, an internal surface of the ribbed cone 440, to extend through opposing surfaces of the ribbed cone 440, or combinations thereof. As shown in FIG. 7, the ribbed cone 440 is disposed at a distal end of a needle 416, which is configured to be slidable within the catheter 402 of FIG. 8.

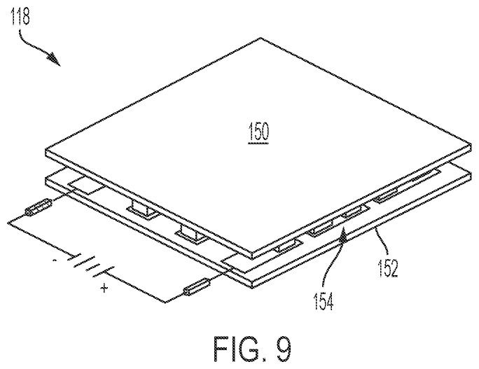

Referring to FIG. 9, an exemplary ETM 118 is illustrated. The ETM 118 includes a first surface component 150 and a second surface component 152 opposite the first surface component 150. The first surface component 150 and the second surface component 152 may be made of ceramic or other suitable conductive material. Electrical junctions as p-n coupled are disposed between the first surface component 150 and the second surface component 152 to energize the surface components 150, 152 through circuitry that sends current to the surface components 150, 152. In embodiments, the ETMs 118 are semiconductor modules including p-n couples 154 as the electrical junctions. The p-n couples are configured so that a supplied first polarity results in heating of one side/surface area such as the first surface component 150 and cooling of the other side such as the second surface component 152, and a supplied second polarity opposite the first polarity results in cooling of the first surface component 150, for example, and heating of the second surface component 152, for example. Such p-n couples 154 connect the electrically connected surface components 150, 152 through an interface including a p-type semiconductor material and an n-type semiconductor material that allows electrical current to pass through in a direction controlled, in the present disclosure, by an associated polarity. In embodiments, circuitry may be employed to control cycling and temperature ranges of the heating and the cooling. By way of example, and not as a limitation, the first surface component 150 may be heated to a temperature from about 45 degrees Celsius to about 50 degrees Celsius while the second surface component 152 is cooled. In an embodiment, the second surface component may be cooled to about −10 degrees Celsius. Then, the polarity may be reversed to cause the first surface component to cool to about −10 degrees Celsius and the second surface component 152 to heat. In an embodiment, the second surface component 152 may be heated to a temperature from about 45 degrees Celsius to about 50 degrees Celsius. The polarity may then be reversed again to heat the first surface component 150 to a temperature from about 45 degrees Celsius to about 50 degrees Celsius while the second surface component 152 is cooled. The polarity may be reversed any number of times sufficient to provide the desired clinical outcome. The polarity reversals may occur with a frequency of a time sequence cycle as determined by the operator to provide the desired clinical outcome. In example embodiments, the polarity reversals may occur at a frequency of the time sequence cycle defined by a switching period of approximately 2 seconds between each polarity reversal.

The tumor ablation probe devices described herein include a controller circuit configured to switch, such as via a relay or switch board, input power polarity to be directed to the ETMs 118 to control the temperature profile through controlled repeatedly alternation between heating and cooling cycles. The controller circuit is configured to control a pre-determined cycling of heating and cooling on the ETMs 118 that are introduced to a tumor site to result in a desired clinical outcome such as tissue/tumor necrosis of the area the ETMs 118 contact through the heating/cooling cycle switching, for example. The controller circuit may be pre-programmed or manually controlled to switch the input voltage polarity that is delivered to the ETMs 118 to result in a corresponding pre-determined cycling of heating and cooling on the opposing surface components 150, 152 of the ETMs 118. The ETMs 118 may be individually controlled and set to output different temperatures at each ETM 118 based on a received input voltage polarity supplied by the controller circuit. Thus, tumor ablation within predetermine threshold ranges of a desired area of a tumor may be achieved over temperature profiles of the ETMs 118 to obtain tumor ablation at tumor margins within the predetermine threshold ranges with respect to specific tumor locations, resulting in controlled localized tumor ablation at controlled tumor margins.

In an embodiment, a switch is configured cause a change in a polarity of a provided voltage. In an embodiment, a positive +5V input, for example, may be switched to a negative −5V input, for example, after a controlled time sequence cycle. It is contemplated and within the scope of this disclosure that other voltage magnitudes are possible. It is further contemplated and within the scope of this disclosure that a switch in polarity may switch between different voltage magnitudes rather than opposing polarities of the same voltage magnitude. A resulting temperature of the first surface component 150 or the second surface component 152 of the ETM 118 is at least partially based on the voltage magnitude. The controlled time sequence cycle may be in a range of about every 2 seconds to about every 5 seconds. In such a scenario, the positive +5V causes the ETM 118 to heat the first surface component 150 to provide heating to the tumor and causes the ETM 118 to heat the second surface component 152 to provide cooling to the tumor, while the negative −5V causes the ETM 118 to cool the first surface component 150 to provide cooling to the tumor and causes the ETM 118 to heat the second surface component 152 to provide heating to the tumor.

The tumor ablation probe devices 100, 200, 300, 400 as described herein may include a probe arm such as a needle, catheter, or a hypotube. The various components of the tumor ablation probe devices, such as the probes 102, 202, 402 and probe arms 116, 216, 316, 416 described herein may be made of a metal, a metal alloy, a polymer, a 3D printed material, or combinations thereof. A base probe of the tumor ablation probe device may include multiple tiers of ETM 118 branches along a length and/or around a circumference, such as shown for the probe arms 116 of FIG. 1. The tumor ablation probe device may be disposable or non-disposable. The tumor ablation probe device may be battery powered such that power is provided via an internal battery and/or powered via an external electrical component to supply voltage that is electronically coupled to the tumor ablation probe device.

The tumor ablation probe devices as described herein are configured to precisely control an amount of heat generated as well as to provide a rapid heating/cooling cycling. Such configurations enhance tumor necrosis while maintaining excellent tumor margins. For instance, the area of the tissue of the tumor to which such rapid heating/cooling is being applied as described herein via a respective ETM 118 is affected based on a surface area of the first surface component 150 or the second surface component 152 of the ETM 118 providing said heating/cooling while being disposed within and flush against said affected tissue. An amount or magnitude as well as polarity of the voltage supplied to the first surface component 150 or the second surface component 150 of a respective ETM 118 as well as a configuration of one or more p-n couples disposed between the first surface component 150 and the second surface component 150 of the respective ETM 118 as described herein are parameters contributing to the rapid heating/cooling cycling to cause tumor necrosis within desired tumor margins. Thus, the devices described herein provide precision temperature control through control of at least a magnitude and direction of an applied voltage to the ETMs 118 of the devices and increased accuracy of a lethal tumor necrosis zone area as a result of such precision temperature control. The resulting area of the lethal tumor necrosis zone is at least in part determined by the temperature profile established by the precision temperature control, which is controlled by a magnitude and polarity of an applied voltage to the ETMs 118 during a time sequence cycle and includes a repeated alternation between voltage polarities. The controlled temperature profile is applied to a tumor via the one or more ETMs 118 as described herein to accurately cause the lethal tumor necrosis in a zone area within precise tumor margins. Such increased accuracy more effectively ablates a tumor within in a desired zone area while not ablating undesired areas that may be disposed near the desired zone area. The increased precision permits for reproducibility of such results within tight error margins.

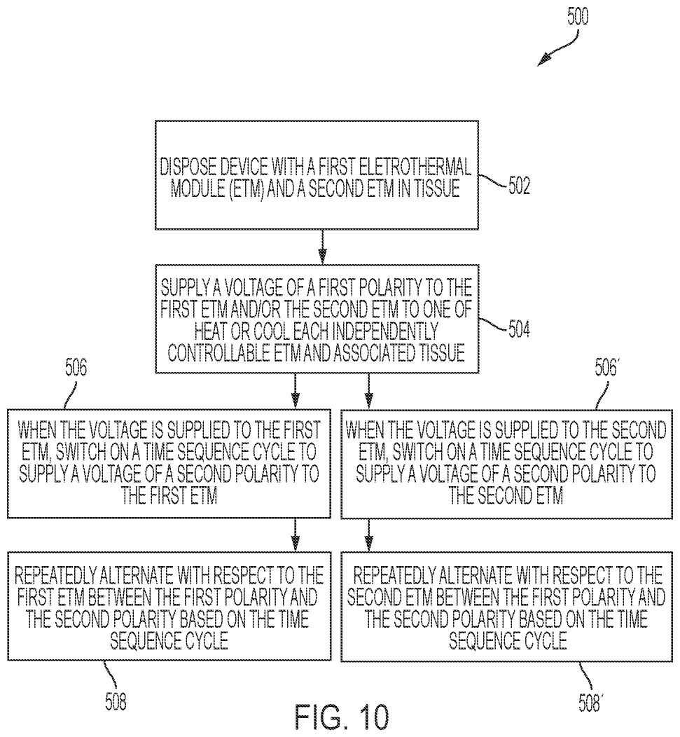

FIG. 10 illustrates a process 500 to use with the tumor ablation probe devices 100, 100′, 200, 300, 400 described herein with reference to FIGS. 1-9. The process 500 includes a method for tumor ablation with controlled precision of a temperature profile utilizing a tumor ablation probe device 100, 100′, 200, 300, 400. In block 502, a distal end 106, 206, 306, 406 of the tumor ablation probe device 100, 100′, 200, 300, 400 is disposed in a tissue 124. By way of example and not as a limitation, the distal end 106, 206, 306, 406 includes, or proximally includes, at least two electrothermal modules (ETMs) 118 of a plurality of ETMs on a device surface, each ETM 118A and 118B (FIG. 1) including a first surface component 150 and a second surface component 152 (FIG. 9) opposite and electrically connected to the first surface component 150.

In block 504, one of a first voltage of a first polarity and a second voltage of a second polarity opposite the first polarity is supplied via a circuit controller communicatively coupled to the tumor ablation probe device 100, 100′, 200, 300, 400 to at least one ETM 118A or 118B of the plurality of ETMs 118. In embodiments, when the first polarity is supplied, the at least one ETM heats the first surface component 150 and cools the second surface component 152, and when the second polarity is supplied, the at least one ETM cools the first surface component 150 and heats the second surface component 152. Each ETM 118 may be configured for independent control by the circuit controller.

In blocks 506, 506′, the other of the first voltage of the first polarity and the second voltage of the second polarity is supplied via the circuit controller to at least one ETM 118A or 118B of the plurality of ETMs 118 to switch the polarity for a repeated alternation between polarities based on a time sequence cycle. In blocks 508, 508′ the voltage is switched with respect to the respective first or second ETM 118A, 118B with the other of the first voltage of the first polarity and the second voltage of the second polarity based on the time sequence cycle. In an embodiment, the time sequence cycle may include a first time sequence cycle associated with a heating stage of the ETM 118 and a second time sequence cycle associated with the cooling stage of the ETM 118 and different from the first time sequence cycle. In either embodiment, whether the first time sequence cycle and the second tiem sequence cycle are the same or different, the process 500 involves switching between the first polarity and the second polarity via the circuit controller based on the time sequence cycle. As a non-limiting example, the time sequence cycle is in a range of about 2 seconds to about 5 seconds, and when the first polarity is supplied, the at least one ETM 118A heats heat the first surface component 150 to a range of about 45 degrees Celsius and about 50 degrees Celsius and cools the second surface component 152. The second surface component 152 may be cooled to about −10 degrees Celsius. When the second polarity is supplied, the at least one ETM 11A cools the first surface component 150 to about −10 degrees Celsius and heats the second surface component 152. The second surface component 152 may be heated to the range between about 45 degrees Celsius and about 50 degrees Celsius.

The distal end 106, 206, 306, 406 may include, or proximally include, at least one ETM 118A on a first probe arm 116A and at least one ETM 118B on a second probe arm 116B (FIG. 1), and each of the first probe arm 116A and the second probe arm 116B may be configured for independent control by the circuit controller. In embodiments, the ETMs 118 on a same probe arm 116 may be independently controlled and/or ETMs 118 on different probe arms 116, such as the first and second probe arms 116A, 116B, may be independently controlled. The first polarity may be positive such that the second polarity is negative. Alternatively, the first polarity may be negative such that the second polarity is positive. The first voltage may be equal to the second voltage, or the first voltage may be different from the second voltage.

In embodiments, one of the first voltage of the first polarity and the second voltage of the second polarity is supplied to one ETM 118A, 118B of the plurality of ETMs 118 as a voltage-supplied ETM and not to the other ETM 118A, 118B as a voltage-deprived ETM. Thus, the supplied ETM heats or cools a respective first or second surface component 150, 152 based on the polarity of the voltage supplied as described herein while the voltage-deprive ETM neither provides heating nor cooling to a respective first or second surface component 150, 152.

The first surface component 150 may be electrically connected to the second surface component 152 through a p-n couple 154. The p-n couple 154 includes a p-type semiconductor in which charge carriers in the material are positive “holes” and an n-type semiconductor material in which charge carriers in the material are negative electrons. Current flow may be controlled in a direction based on an applied voltage polarity. By way of example, and not as a limitation, the p-n couple 154 includes an n-type semiconductor material joined to a p-type semiconductor material such that a positive voltage causes an ETM 118 to which the positive voltage is supplied to heat on one of the first surface component 150 and the second surface component 152 while a negative voltage supplied to the ETM 118 alternatively causes the ETM 118 to heat to the other of the first surface component 150 and the second surface component 152.

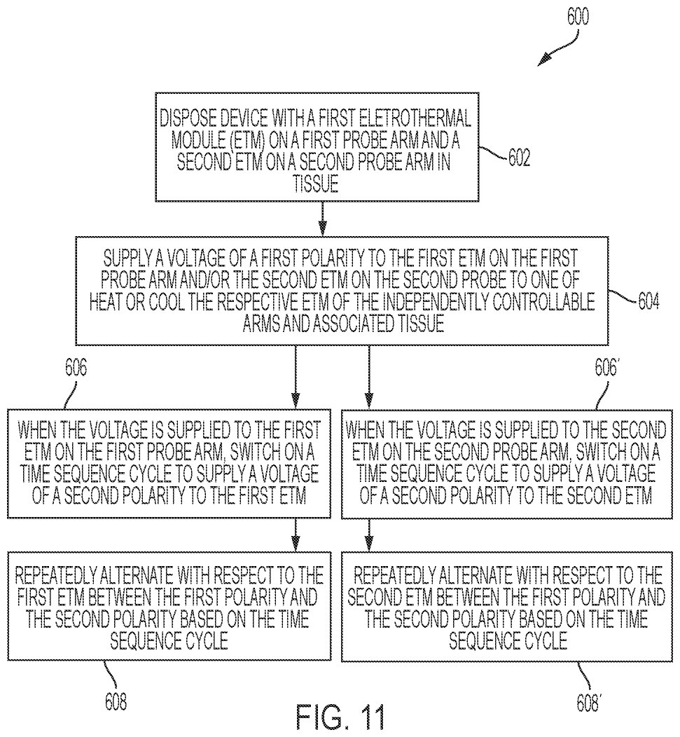

FIG. 11 illustrates a process 600 to use with the tumor ablation probe devices 100, 100′, 200, 300, 400 described herein. The process 500 includes another method for tumor ablation with controlled precision of a temperature profile utilizing a tumor ablation probe device 100, 100′, 200, 300, 400. In block 602, a distal end 106, 206, 306, 406 of the tumor ablation probe device 100, 100′, 200, 300, 400 is disposed in a tissue 124. By way of example and not as a limitation, the distal end 106, 206, 306, 406 includes at least one ETM 118A on a first probe arm 116A and at least one ETM 118B on a second probe arm 116B, each ETM 118A and 118B (FIG. 1) including a first surface component 150 and a second surface component 152 (FIG. 9) opposite and electrically connected to the first surface component 150.

In block 604, one of a first voltage of a first polarity and a second voltage of a second polarity opposite the first polarity is supplied via a circuit controller communicatively coupled to the tumor ablation probe device 100, 100′, 200, 300, 400 to the at least one ETM 118A on the first probe arm 116A, the at least one ETM 118B on the second probe arm 116B, or both. In embodiments, the first polarity is configured to heat the first surface component 150 and cool the second surface component 152, and the second polarity is configured to cool the first surface component 150 and heat the second surface component 152. Each probe arm 116, such as the first probe arm 116A and the second probe arm 116B, may be configured for independent control by the circuit controller.

In blocks 606, 606′, the other of the first voltage of the first polarity and the second voltage of the second polarity is supplied via the circuit controller to the at least one ETM 118A on the first probe arm 116A, the at least one ETM 118B on the second probe arm 116B, or both as one or more voltage-supplied ETMs, to repeatedly alter and switch the polarity based on a time sequence cycle. In blocks 608, 608′ the voltage is switched again with respect to the respective first or second ETM 118A, 118B with the other of the first voltage of the first polarity and the second voltage of the second polarity using the time sequence cycle. The, via the circuit controller, the process 600 involves a repeated alteration between the first polarity and the second polarity using the time sequence cycle.

In embodiments, each ETM 118 may be configured for independent control by the circuit controller, the first polarity is one of positive and negative and the second polarity is the other of positive and negative, and/or the first voltage may be equal to or different from the second voltage.

Referring to FIG. 12, a system 700 for implementing a computer and software-based method to utilize the tumor ablation probe devices of FIGS. 1-9 and the processes of FIGS. 10-11 is illustrated and may be implemented along with using a graphical user interface (GUI) that is accessible at a user workstation (e.g., a computer 724), for example. The system 700 includes a communication path 702, one or more processors 704, a memory component 706, a tumor ablation probe device 712 that may be any of the tumor ablation probe devices 100, 100′, 200, 300, 400 described herein, a storage or database 714, a switching component 716, a network interface hardware 718, a server 720, a network 722, and at least one computer 724. The various components of the system 700 and the interaction thereof will be described in detail below.

In some embodiments, the system 700 is implemented using a wide area network (WAN) or network 722, such as an intranet or the Internet, or other wired or wireless communication network that may include a cloud computing-based network configuration. The workstation computer 724 may include digital systems and other devices permitting connection to and navigation of the network. The lines depicted in FIG. 7 indicate communication rather than physical connections between the various components.

As noted above, the system 700 includes the communication path 702. The communication path 702 may be formed from any medium that is capable of transmitting a signal such as, for example, conductive wires, conductive traces, optical waveguides, or other media capable of transmitting signals, or from a combination of media capable of transmitting signals. The communication path 702 communicatively couples the various components of the system 700. As used herein, the term “communicatively coupled” means that coupled components are capable of exchanging data signals with one another such as, for example, electrical signals via conductive medium, electromagnetic signals via air, optical signals via optical waveguides, or other data signals via a corresponding data signal exchange medium.

As previously described, the system 700 includes the processor 704. The processor 704 can be any device capable of executing machine readable instructions. Accordingly, the processor 704 may be a controller such as the circuit controller described herein, an integrated circuit, a microchip, a computer, or any other computing device. The processor 704 is communicatively coupled to the other components of the system 700 by the communication path 702. Accordingly, the communication path 702 may communicatively couple any number of processors with one another, and allow the modules coupled to the communication path 302 to operate in a distributed computing environment. Specifically, each of the modules can operate as a node that may send and/or receive data. The processor 704 may process the input signals received from the system modules and/or extract information from such signals.

As previously described, the system 700 includes the memory component 706 coupled to the communication path 702 and communicatively coupled to the processor 704. The memory component 706 may be a non-transitory computer readable medium or non-transitory computer readable memory and may be configured as a nonvolatile computer readable medium. The memory component 706 may comprise RAM, ROM, flash memories, hard drives, or any device capable of storing machine readable instructions such that the machine readable instructions can be accessed and executed by the processor 704. The machine readable instructions may comprise logic or algorithm(s) written in any programming language such as, for example, machine language that may be directly executed by the processor, or assembly language, object-oriented programming (OOP), scripting languages, microcode, etc., that may be compiled or assembled into machine readable instructions and stored on the memory component 706. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. In embodiments, the system 700 may include the processor 704 communicatively coupled to the memory component 706 that stores instructions that, when executed by the processor 704, cause the processor to perform one or more functions as described herein.

Still referring to FIG. 12, as previously described, the system 700 comprises the display such as a GUI on a screen of the computer 724 for providing visual output such as, for example, information, graphical reports, messages, or a combination thereof. The computer 724 may include one or more computing devices across platforms, or may be communicatively coupled to devices across platforms, such as mobile smart devices including smartphones, tablets, laptops, and/or other smart devices. The display can include any medium capable of transmitting an optical output such as, for example, a cathode ray tube, light emitting diodes, a liquid crystal display, a plasma display, or other optical output transmission mediums. Additionally, it is noted that the display or the computer 724 can include at least one of the processor 704 and the memory component 706. While the system 700 is illustrated as a single, integrated system in FIG. 12, in other embodiments, the systems can be independent systems.

The system 700 comprises the tumor ablation probe device 712 as described herein to cause tumor necrosis via one or more ETMs 118 and the switching component 716 to cause the heating and cooling cycling to power the ETMs 118 to act to employ thermal energy to the tissue through a time sequence cycle causing a repeated alteration between voltage polarities to affect a thermal profile and thus to enhance tumor necrosis. The tumor ablation probe device 712 and the switching component 716 are coupled to the communication path 702 and communicatively coupled to the processor 704. As will be described in further detail below, the processor 704 may process the input signals received from the system modules and/or extract information from such signals.

The system 700 includes the network interface hardware 718 for communicatively coupling the system 700 with a computer network such as network 722. The network interface hardware 718 is coupled to the communication path 702 such that the communication path 702 communicatively couples the network interface hardware 718 to other modules of the system 700. The network interface hardware 718 can be any device capable of transmitting and/or receiving data via a wireless network. Accordingly, the network interface hardware 718 can include a communication transceiver for sending and/or receiving data according to any wireless communication standard. For example, the network interface hardware 718 can include a chipset (e.g., antenna, processors, machine readable instructions, etc.) to communicate over wired and/or wireless computer networks such as, for example, wireless fidelity (Wi-Fi), WIMAX, BLUETOOTH, IRDA, WIRELESS USB, Z-WAVE, ZIGBEE, or other chipsets.

Still referring to FIG. 12, data from various applications running on computer 724 can be provided from the computer 724 to the system 700 via the network interface hardware 318.

The computer 724 can be any device having hardware (e.g., chipsets, processors, memory, etc.) for communicatively coupling with the network interface hardware 718 and a network 722. Specifically, the computer 724 can include an input device having an antenna for communicating over one or more of the wireless computer networks described above.

The network 722 can include any wired and/or wireless network such as, for example, wide area networks, metropolitan area networks, the Internet, an intranet, the cloud, satellite networks, or other networks. Accordingly, the network 722 can be utilized as a wireless access point by the computer 724 to access one or more servers (e.g., a server 720). The server 720 and any additional servers generally include processors, memory, and chipset for delivering resources via the network 722. Resources can include providing, for example, processing, storage, software, and information from the server 720 to the system 700 via the network 722. Additionally, it is noted that the server 720 and any additional servers can share resources with one another over the network 722 such as, for example, via the wired portion of the network, the wireless portion of the network, or combinations thereof.

### Items Listing

Item 1. A system for tumor ablation with controlled precision of a temperature profile utilizing a tumor ablation probe device may include the tumor ablation probe device including a distal end, the distal end comprising a plurality of electrothermal modules (ETMs) proximally disposed on a device surface, each ETM including a first surface component and a second surface component opposite and electrically connected to the first surface component. The system may further include a circuit controller communicatively coupled to the tumor ablation probe device and a non-transitory computer storage medium. The non-transitory computer storage medium stores instructions that, when executed by the circuit controller, may cause the system to supply, via the circuit controller, one of a first voltage of a first polarity and a second voltage of a second polarity opposite the first polarity to at least one ETM of the plurality of ETMs, and repeatedly alternate, via the circuit controller, between the first polarity and the second polarity using a time sequence cycle. When the first polarity is supplied, the at least one ETM heats the first surface component and cools the second surface component. When the second polarity is supplied, the at least one ETM cools the first surface component and heats the second surface component, and each ETM may be configured for independent control by the circuit controller.

Item 2. The system of Item 1, the non-transitory computer storage medium stores further instructions that, when executed by the circuit controller, cause the system to supply one of the first voltage of the first polarity and the second voltage of the second polarity to the at least one ETM of the plurality of ETMs and not to at least one other ETM of the plurality of ETMs.

Item 3. The system of any of Items 2-3, wherein the distal end includes the at least one ETM of the plurality of ETMs on a first probe arm and at least one ETM of the plurality of ETMs on a second probe arm, and each of the first probe arm and the second probe arm is configured for independent control by the circuit controller.

Item 4. The system of any of Items 1-3, wherein the first polarity is positive and the second polarity is negative.

Item 5. The system of any of Items 1-3, wherein the first polarity is negative and the second polarity is positive.

Item 6. The system of any of Items 1-5, wherein the first voltage is equal to the second voltage.

Item 7. The system of any of Items 1-5, wherein the first voltage is different from the second voltage.

Item 8. The system of any of Items 1-7, wherein the time sequence cycle is from about 2 seconds to about 5 seconds.

Item 9. The system of any of Items 1-8, wherein when the first polarity is supplied, the at least one ETM heats the first surface component to a range from about 45 degrees Celsius to about 50 degrees Celsius, and when the second polarity is supplied, the at least one ETM cools the first surface component to about −10 degrees Celsius.

Item 10. The system of any of Items 1-9, wherein the first surface component of each ETM is electrically connected to the second surface component of each respective ETM through a p-n couple.

Item 11. A method for tumor ablation with controlled precision of a temperature profile utilizing a tumor ablation probe device may include disposing a distal end of the tumor ablation probe device in a tissue, the distal end comprising a plurality of electrothermal modules (ETMs) proximally disposed on a device surface, each ETM including a first surface component and a second surface component opposite and electrically connected to the first surface component. The method may further include supplying, via a circuit controller communicatively coupled to the tumor ablation probe device, one of a first voltage of a first polarity and a second voltage of a second polarity opposite the first polarity to at least one ETM of the plurality of ETMs, and repeatedly alternating, via the circuit controller, between the first polarity and the second polarity based on a time sequence cycle. When the first polarity is supplied, the at least one ETM heats the first surface component and cools the second surface component. When the second polarity is supplied, the at least one ETM cools the first surface component and heats the second surface component, and each ETM may be configured for independent control by the circuit controller.

Item 12. The method of Item 11, wherein the one of the first voltage of the first polarity and the second voltage of the second polarity is supplied to the at least one ETM of the plurality of ETMs and not to at least one other ETM of the plurality of ETMs.

Item 13. The method of any of Items 11-12, wherein the distal end includes the at least one ETM of the plurality of ETMs on a first probe arm and at least one other ETM of the plurality of ETMs on a second probe arm, and each of the first probe arm and the second probe arm is configured for independent control by the circuit controller.

Item 14. A method for tumor ablation with controlled precision of a temperature profile utilizing a tumor ablation probe device may include disposing a distal end of the tumor ablation probe device in a tissue, the distal end comprising at least one electrothermal module (ETM) on a first probe arm and at least one ETM on a second probe arm, supplying, via a circuit controller communicatively coupled to the tumor ablation probe device, one of a first voltage of a first polarity and a second voltage of a second polarity opposite the first polarity to the at least one ETM on the first probe arm, the at least one ETM on the second probe arm, or both as one or more voltage-supplied ETMs, and repeatedly alternating, via the circuit controller, between the first polarity and the second polarity using a time sequence cycle. Each ETM may include a first surface component and a second surface component opposite and electrically connected to the first surface component. When the first polarity is supplied, the one or more voltage-supplied ETMs respectively heats the first surface component and cools the second surface component. When the second polarity is supplied, the one or more voltage-supplied ETMs respectively cools the first surface component and heats the second surface component, and each of the first probe arm and the second probe arm may be configured for independent control by the circuit controller.

Item 15. The method of Item 14, wherein when the one of the first voltage of the first polarity and the second voltage of the second polarity is supplied to one of the at least one ETM on the first probe arm and the at least one ETM on the second probe arm and not to the other of the at least one ETM on the first probe arm and the at least one ETM on the second probe arm.

Item 16. The method of any of Items 14-15, wherein each ETM is configured for independent control by the circuit controller.

Item 17. The method of any of Items 14-16, wherein the first polarity is positive and the second polarity is negative.

Item 18. The method of any of Items 14-16, wherein the first polarity is negative and the second polarity is positive.

Item 19. The method of any of Items 14-18, wherein the first voltage is equal to the second voltage.

Item 20. The method of any of Items 11-15, wherein the first voltage is different from the second voltage.

It is noted that the terms “substantially” and “about” and “approximately” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system for tumor ablation with controlled precision of a temperature profile utilizing a tumor ablation probe device, the system comprising:
the tumor ablation probe device including a distal end, the distal end comprising a plurality of electrothermal modules (ETMs) proximally disposed on a device surface, each ETM including a first surface component and a second surface component opposite and electrically connected to the first surface component; and
a circuit controller communicatively coupled to the tumor ablation probe device and a non-transitory computer storage medium,
wherein the non-transitory computer storage medium stores instructions that, when executed by the circuit controller, cause the system to:
supply, via the circuit controller, one of a first voltage of a first polarity and a second voltage of a second polarity opposite the first polarity to at least one ETM of the plurality of ETMs, wherein when the first polarity is supplied, the at least one ETM heats the first surface component and cools the second surface component, and when the second polarity is supplied, the at least one ETM cools the first surface component and heats the second surface component; and
repeatedly alternate, via the circuit controller, between the first polarity and the second polarity based on a time sequence cycle, wherein each ETM is configured for independent control by the circuit controller.

2. The system of claim 1, the non-transitory computer storage medium stores further instructions that, when executed by the circuit controller, cause the system to supply one of the first voltage of the first polarity and the second voltage of the second polarity to the at least one ETM of the plurality of ETMs and not to at least one other ETM of the plurality of ETMs.

3. The system of claim 1, wherein the distal end includes the at least one ETM of the plurality of ETMs on a first probe arm and at least other one ETM of the plurality of ETMs on a second probe arm, and each of the first probe arm and the second probe arm is configured for independent control by the circuit controller.

4. The system of claim 1, wherein the first polarity is positive and the second polarity is negative.

5. The system of claim 1, wherein the first polarity is negative and the second polarity is positive.

6. The system of claim 1, wherein the first voltage is equal to the second voltage.

7. The system of claim 1, wherein the first voltage is different from the second voltage.

8. The system of claim 1, wherein the time sequence cycle is from about 2 seconds to about 5 seconds.

9. The system of claim 1, wherein when the first polarity is supplied, the at least one ETM heats the first surface component to a range from about 45 degrees Celsius to about 50 degrees Celsius, and when the second polarity is supplied, the at least one ETM cools the first surface component to about −10 degrees Celsius.

10. The system of claim 1, wherein the first surface component of each ETM is electrically connected to the second surface component of each respective ETM through a p-n couple.

11. A method for tumor ablation with controlled precision of a temperature profile utilizing a tumor ablation probe device, the method comprising:
disposing a distal end of the tumor ablation probe device in a tissue, the distal end comprising a plurality of electrothermal modules (ETMs) proximally disposed on a device surface, each ETM including a first surface component and a second surface component opposite and electrically connected to the first surface component; and
supplying, via a circuit controller communicatively coupled to the tumor ablation probe device, one of a first voltage of a first polarity and a second voltage of a second polarity opposite the first polarity to at least one ETM of the plurality of ETMs, wherein when the first polarity is supplied, the at least one ETM heats the first surface component and cools the second surface component, and wherein when the second polarity is supplied, the at least one ETM cools the first surface component and heats the second surface component; and
repeatedly alternating, via the circuit controller, between the first polarity and the second polarity using a time sequence cycle, wherein each ETM is configured for independent control by the circuit controller.

12. The method of claim 11, wherein the one of the first voltage of the first polarity and the second voltage of the second polarity is supplied to the at least one ETM of the plurality of ETMs and not to at least one other ETM of the plurality of ETMs.

13. The method of claim 11, wherein the distal end includes the at least one ETM of the plurality of ETMs on a first probe arm and at least one other ETM of the plurality of ETMs on a second probe arm, and each of the first probe arm and the second probe arm is configured for independent control by the circuit controller.

14. A method for tumor ablation with controlled precision of a temperature profile utilizing a tumor ablation probe device, the method comprising:
disposing a distal end of the tumor ablation probe device in a tissue, the distal end comprising at least one electrothermal module (ETM) on a first probe arm and at least one ETM on a second probe arm, each ETM including a first surface component and a second surface component opposite and electrically connected to the first surface component;
supplying, via a circuit controller communicatively coupled to the tumor ablation probe device, one of a first voltage of a first polarity and a second voltage of a second polarity opposite the first polarity to the at least one ETM on the first probe arm, the at least one ETM on the second probe arm, or both as one or more voltage-supplied ETMs, wherein when the first polarity is supplied, the one or more voltage-supplied ETMs respectively heats the first surface component and cools the second surface component, and when the second polarity is supplied, the one or more voltage-supplied ETMs cools the first surface component and heats the second surface component; and
repeatedly alternating, via the circuit controller, between the first polarity and the second polarity using a time sequence cycle, wherein each of the first probe arm and the second probe arm is configured for independent control by the circuit controller.

15. The method of claim 14, wherein the one of the first voltage of the first polarity and the second voltage of the second polarity is supplied to one of the at least one ETM on the first probe arm and the at least one ETM on the second probe arm and not to the other of the at least one ETM on the first probe arm and the at least one ETM on the second probe arm.

16. The method of claim 14, wherein each ETM is configured for independent control by the circuit controller.

17. The method of claim 14, wherein the first polarity is positive and the second polarity is negative.

18. The method of claim 14, wherein the first polarity is negative and the second polarity is positive.

19. The method of claim 14, wherein the first voltage is equal to the second voltage.

20. The method of claim 14, wherein the first voltage is different from the second voltage.
